(19) United States
(12) Patent Application Publication
(10) Pub. No.: US 2011/0307037 A1
(43) Pub. Date: Dec. 15, 2011

(54) THERMAL THERAPY FOR PREVENTION AND/OR TREATMENT OF CARDIOVASCULAR DISEASES AND OTHER AILMENTS

(76) Inventor: Tofy Mussivand, Navan (CA)

(21) Appl. No.: 13/003,849

(22) PCT Filed: Apr. 30, 2009
(86) PCT No.: PCT/CA09/00593

§ 371 Date: Apr. 7, 2011

(30) Foreign Application Priority Data
Jul. 15, 2008 (US) 61080993

(51) Int. Cl. A61F 7/00

(52) U.S. Cl. 607/96

(57) ABSTRACT

There is provided a method for treating an ailment in a patient comprising: raising a temperature of at least one portion of the patient's body by an unendangering pre-determined amount; holding an increased temperature for the at least one portion for an unendangering pre-determined time period; and repeating the raising and holding steps until a desired results is achieved.

### CROSS REFERENCE TO RELATED APPLICATIONS

The present disclosure claims priority from U.S. Provisional Patent Application No. 61/080,993, filed Jul. 15, 2008, the entirety of which is hereby incorporated by reference.

### TECHNOLOGICAL FIELD

This disclosure provides a platform approach, methods and devices for prevention and treatment of various diseases including cardiovascular (e.g., hypertension, congestive heart failure, various arrhythmias, etc.) and non-cardiovascular ailments using thermal therapy. In particular, this disclosure provides thermal therapy for prevention and/or treatment of cardiovascular and non-cardiovascular diseases such as arrhythmias, heart failure and hypertension.

### BACKGROUND

Hospitalization of patients with heart failure has been steadily increasing despite modern advances in the treatment and prevention of cardiovascular diseases (1). Heart failure is a frequent cause of disability and loss of independence (2). Furthermore, heart failure has been associated with long-term survival rates that are worse than those for many types of cancer, with the exception of lung cancer (3). The heart failure epidemic has thus emerged as a critical priority for national governments (4). In the US alone, more than 5.2 million adults have congestive heart failure with an annual cost of approximately $33.2 billion in 2007 (5). It has been estimated that approximately 1% of the health care budget in developed countries is now spent on the diagnosis and management of patients with heart failure (2). Heart failure is also considered a global priority. The World Health Organization (WHO) estimates that cardiovascular disease including heart failure is now responsible for one third of all deaths globally. Furthermore, WHO has forecasted that by 2010 cardiovascular disease will be the leading cause of death in developing countries (6). This means that even if very effective drugs and devices become available for heart failure, the cost will remain unaffordable (particularly in developing countries). Some common cardiovascular ailments are described below.

### Hypertension (High Blood Pressure)

Blood pressure is the amount of blood the heart pumps and the amount of resistance to blood flow that exists in the vessels. The more blood the heart pumps and the narrower the arteries, the higher the blood pressure. Major aspects of high blood pressure are indicated below [60].

Symptoms: Most people with high blood pressure have no signs or symptoms, even if blood pressure readings reach dangerously high levels.

Causes: In 90-95% of high blood pressure cases, there is no identifiable cause. This type of high blood pressure, called essential hypertension or primary hypertension, tends to develop gradually over many years.

The other 5-10% of high blood pressure cases are caused by an underlying condition. This type of high blood pressure, called secondary hypertension, tends to appear suddenly and cause higher blood pressure than does primary hypertension. Various conditions may lead to secondary hypertension, including kidney abnormalities, tumors of the adrenal gland or certain congenital heart defects.

Certain medications (including birth control pills, cold remedies, decongestants, over the counter pain relievers and some prescription drugs) also may cause secondary hypertension.

### Guidelines

Normal Blood Pressure: Blood pressure is normal if it is below 120/80 mmHg, but some data indicates that 115/75 mmHg should be the gold standard. Once blood pressure rises above 115/75 mmHg, the risk of cardiovascular disease begins to increase.

Prehypertension: The prehypertension stage is when there is a systolic pressure ranging from 120 to 130 or a diastolic pressure ranging from 80 to 89. Prehypertension tends to get worse over time. Within four years of being diagnosed with prehypertension, nearly one in three adults, aged 35 to 64 and nearly one in two adults aged 65 or older, progress to definite high blood pressure.

Stage 1 hypertension: Stage 1 hypertension is a systolic pressure ranging from 140 to 159 or a diastolic pressure ranging from 90 to 99.

Stage 2 hypertension: The most severe hypertension, stage 2 hypertension, is a systolic pressure of 160 or higher or a diastolic pressure of 100 or higher.

Both numbers in a blood pressure reading are important. But after the age of 50, the systolic reading is even more significant. Isolated systolic hypertension (ISH), which is when diastolic pressure is normal but systolic pressure is high, is the most common type of high blood pressure among people older than 50.

A single high blood pressure reading usually is not enough for a diagnosis. Because blood pressure normally varies throughout the day and sometimes significantly during visits to the doctor, diagnosis is based on more than one reading taken on more than one occasion. The doctor may ask for a record of your blood pressure at home and at work to provide additional information.

If there is any type of high blood pressure, the doctor may recommend routine tests such as a urine test (urinalysis), blood tests, and an electrocardiogram (ECG). An ECG measures the heart's electrical activity. More extensive testing is not usually needed.

### Complications

Excessive blood pressure on the artery walls may damage vital organs. The higher the blood pressure and the longer it goes uncontrolled, the greater the damage.

Uncontrolled high blood pressure may lead to:

Damage to the arteries: This may result in hardening and thickening of the arteries (atherosclerosis), which may lead to a heart attack or other complications. An enlarged, bulging blood vessel, an aneurysm, is also possible.

Heart failure. In order to pump blood against the higher pressure in the vessels, the heart muscle must thicken. Eventually, the thickened muscle may have a hard time pumping enough blood to meet the body's needs, which may lead to heart failure.

A blocked or ruptured blood vessel in the brain. This may lead to stroke and death.

Weakened and narrowed blood vessels in the kidneys. This may prevent these organs from functioning normally.

Thickened, narrowed or torn blood vessels in the eyes. This may result in vision loss.

Metabolic syndrome. High blood pressure is associated with other components of metabolic syndrome. This syndrome is a cluster of disorders of the body's metabolism, including elevated waist circumference, high triglycerides, low high density lipoprotein (HDL) or “good” cholesterol, high levels blood pressure and high levels of insulin.

Brain damage: Uncontrolled high blood pressure may affect the ability to think, remember and learn. Cognitive impairment and dementia are common among people with high blood pressure.

### High Blood Pressure Treatments:

The major types of medication used to control high blood pressure include:

Thiazide diuretics. These medications act on the kidneys to help the body eliminate sodium and water, thereby reducing blood volume. Thiazide diuretics are often the first, but not the only, choice in high blood pressure medications. In a 2006 study, diuretics were a key factor in preventing heart failure associated with high blood pressure.

Beta blockers. These medications reduce the workload on the heart, causing the heart to beat slower and with less force. These medications also reduce nerve impulses to blood vessels, reducing the effects of natural chemicals that narrow blood vessels.

Angiotensin converting enzyme (ACE) inhibitors. These medications help relax blood vessels by blocking the formation of a natural chemical that narrows blood vessels. ACE inhibitors may be especially important in treating high blood pressure in people with coronary artery disease, heart failure or kidney failure. Like beta blockers, ACE inhibitors do not work as well in African Americans when prescribed alone, but they are effective when combined with thiazide diuretics

Angiotensin II receptor blockers. These medications help relax blood vessels by blocking the action, not the formation, of a natural chemical that narrows blood vessels. Like ACE inhibitors, angiotensin II receptor blockers are often useful for people with coronary artery disease, heart failure and kidney failure.

Calcium channel blockers. These medications help relax the muscles in blood vessels and some slow the heart rate. Calcium channel blockers may work better for African Americans than do ACE inhibitors or beta blockers alone. A word of caution for grapefruit lovers, though. Grapefruit juice interacts with some calcium channel blockers, increasing blood levels of the medication and putting you at higher risk of side effects. Researchers have identified the substance in grapefruit juice that causes the potentially dangerous interaction which may one day lead to commercial grapefruit juices that do not pose a risk of interaction.

Renin inhibitors. A new drug, Tekturna (aliskiren), is a renin inhibitor. Renin is an enzyme produced by the kidneys that starts a cascade of chemical steps which increases blood pressure. Tekturna works by reducing the ability of renin to begin this process. Tekturna acts earlier in the body's blood pressure regulation process than most other blood pressure medications. It also may be used in conjunction with the other major classes of blood pressure drugs to improve their actions. Tekturna may be used alone, but it is more effective when used in combination with existing high blood pressure medications such as water pills (diuretics). Tekturna's effects on blood pressure last more than 25 hours so it may be taken once daily in oral tablet form.

Alpha beta blockers. In addition to reducing nerve impulses to blood vessels, alpha beta blockers slow the heartbeat to reduce the amount of blood that must be pumped through the vessels.

Central acting agents. These medications prevent the brain from signaling the nervous system to increase the heart rate and narrow the blood vessels.

Vasodilators. These medications work directly on the muscles in the walls of arteries, preventing the muscles from tightening and arteries from narrowing.

Resistant hypertension: When blood pressure is difficult to control. If blood pressure remains high despite taking at least three medications from different classes of antihypertensive drugs, one of which is a diuretic, a resistant hypertension may exist where hypertension is resistant to treatment.

### Arrhythmias

Arrhythmias are the leading cause of cardiac death. They are irregular heart rhythms that occur in patients of all ages all over the world, though especially in older ages. Currently, the majority of patients are treated with anti-arrhythmic drugs many of which produce side effects and some, themselves, may contribute to the severity of the arrhythmias. In addition to drugs, implantable cardiac defibrillators (ICDs) are used as treatment devices. Although they have become the gold standard, they are invasive, create painful shocks and are expensive.

Arrhythmia is a disturbance of the heart's natural pulsating rhythm. The cause of arrhythmia varies and may be one or more of the following:

a. Prolonged action potential.

b. Delayed after depolarization.

c. Early after depolarization.

d. Calcium channel disturbance and conditions.

e. Long QT syndrome.

f. Cardiomyopathy.

g. Nutrient and drug impact or effect.

h. Ischemia.

i. Other causes

According to the present disclosure, arrhythmia may be in the form of an atrial and/or ventricular arrhythmia corresponding to the location of beat with respect to the heart muscle. Arrhythmia may be prevented and/or treated with the present methods, whether they are caused by prolonged action potentials and/or other causes such as ischemia and cardiomyopathy.

There are several types of arrhythmias. Each type of arrhythmia may have a different cause. Ventricular arrhythmia may be caused by prolongation of the action potential. For such cases, there are currently two general methods of treatment:

a. Implantable Cardiac Defibrillator (ICD) implantation

b. Use of pharmaceutical agents that shorten the action potential such as Pinacidil or Nexilitine.

Some medical cases use drugs for heart rate regulation such as beta adrenergic drugs (i.e. Isoproterenol).

Pacemakers may also be used to speed up or increase the beat rate of the heart.

If the cause of arrhythmia is ischemia, then general methods of treatment include:

a. Opening of the coronary artery by angioplasty

b Opening of the coronary artery with bypass grafts.

Again, sometimes beta adrenergic antagonistic and other anti-arrhythmic medications are used. For example, Amiodarone. Sometimes, ICDs are used for such arrhythmias a well.

All of the above methods have their own limitations and problems. For example, coronary angioplasty or bypass appears to be, in many cases, incomplete and ineffective because of various problems such as the recurrence of ventricular fibrillation. If anti-arrhythmic agents are used, they may be exasperating as a result of excessive action potential prolongation and sometimes as a result of toxicity (for example, Amiodarone). If beta adrenergic antagonists are used, they may cause a reduction in the contractile forces of the heart.

In summary, to date, there are several methods for ventricular arrhythmias caused by the prolongation of an action potential. These methods are:

1. Use of ICDs

2. Shortening of the action potential by using drugs such as Pinacidil.

3. Increasing heart rate by using pharmaceutical drugs, such as beta-adrenergic agents like Isoproterenol.

4. Increasing beat rate by using a pacing device.

5. Enhancing L-type calcium ions and calcium release from the sarcoplasmic reticulum with beta-adrenergic blocking drugs.

None of the above methods are widely or universally effective or efficient for various reasons including:

a. Patient variation

b. Prolongation of the action potential or non-responsiveness to the pacing device may shorten the action potential prolongation. Some drugs may actually exasperate and cause arrhythmias themselves (such as Isoproterenol). Some agents, such as beta-adrenergic antagonists may reduce the contractility force of the heart, thus indirectly impacting and contributing to the complications.

Another arrhythmia is atrial fibrillation. Atrial fibrillation is associated with heart failure, stroke and mortality. This irregularity in beating may be associated with inflammation, structural and functional abnormalities, prolonged action potential, early after-depolarization in action potential pulses, and delayed depolarization. It may be caused by cellular and molecular abnormalities including, but not limited to, calcium ions and calcium channel abnormalities, cardiac structural and remodeling, long QT syndrome, cardiomyopathy, drugs, nutrients and ischemic causes. The preferred embodiment, as far as arrhythmias, of the present disclosure is the treatment of tachycardia, bradycardia, ventricular fibrillation, atrial fibrillation and other arrhythmic rhythms. Therapies include:

a. Anticoagulation for prevention of stroke

b. Heart rate control

c. Pacing by ICDs

Unfortunately, anti-arrhythmic drugs have a limited effectiveness (about 50%).

### Hypertrophic Disease

Hypertrophic disease consists of cell growth without increase in cell number. Hypertrophy affects various tissues. For example:

a. Hypertension in the form of an increase in smooth muscle cell volume of the blood vessels due to excessive pressure.

b. Any lack of sufficient oxygen and/or nutrients

c. Common and resulting hypertrophic inducing factors at specific sites such as various arrhythmia diseases and/or cardiac disease such as congestive heart failure (cardiomyopathy caused by hypertrophy)

d. Traumas such as hypoxia causing cardiomyocyte increase in volume.

e. Other cellular hypertrophy and inflammation caused by chemical and other stimuli.

### SUMMARY

The present disclosure provides both methods and devices for the prevention and/or treatment of arrhythmias, heart failure, hypertension and other cardiovascular and non-cardiovascular ailments with thermal therapy, in which the administration of multiple drugs may be continued and/or reduced and/or eliminated, typically under the supervision of a licensed physician. These methods and/or systems may be applied to other ailments.

A method for prevention and treatment of arrhythmias, heart failure and hypertension as well as other ailments is disclosed, in which various pathways for reverse remodeling, vasodilatation, increased nitric oxide, heat shock proteins and other mechanisms may be triggered and/or enhanced by an increase in the body's core temperature, all or some of which may lead to the activation of the patient's healing capacity, which may improve conditions, signs and symptoms. In some aspects, there is provided a method for treating or preventing an ailment in a patient comprising: raising a temperature of at least one portion of the patient's body by a pre-determined amount, which may be an unendangering or safe amount; and holding an increased temperature for the at least one portion for a pre-determined time period, which may be an unendangering or safe time period. In some embodiments, raising the temperature and holding the increased temperature may be repeated until the desired result is achieved. In some embodiments, the temperature of the patient's entire body may be raised and the increased temperature may be held for the entire body.

The method may be used in conjunction with medication and/or therapeutic devices for treating or preventing an ailment.

The method may be used for treating or preventing cardiovascular or non-cardiovascular diseases.

### BRIEF DESCRIPTION OF FIGURES

FIG. 1 is a graph illustrating the effect of thermal therapy on ejection fraction (EF), in accordance with some embodiments;

FIG. 2 is a graph illustrating the effect of thermal therapy on cardiac index (CI), in accordance with some embodiments;

FIG. 3 is a graph illustrating the effect of thermal therapy on systemic vascular resistance (SVR), in accordance with some embodiments;

FIG. 4 is a graph illustrating the effect of thermal therapy on neurohormonal function, in accordance with some embodiments;

FIG. 5 is a graph illustrating the effect of thermal therapy on cardiac arrhythmias, in accordance with some embodiments;

FIG. 6 outlines some other effects of thermal therapy, in accordance with some embodiments;

FIG. 7 is a schematic diagram illustrating a NO production and pathway for vasodilation;

FIG. 8 is a graph illustrating the effect of various thermal therapy regimes on ejection fraction in NYHA Class II-IV heart failure patients, in accordance with some embodiments; and

Table 1 lists a summary of the findings of published studies on the clinical effects of thermal therapy in heart failure, in accordance with some embodiments.

### DETAILED DESCRIPTION

The present disclosure may provide a platform approach, methods and devices for stimulating, enhancing and initiating the body's natural healing process.

The search for effective, safe and affordable treatment modalities for various cardiovascular and non-cardiovascular ailments continues. One such potential modality is an increased body core and surface temperature, also referred to as “thermal therapy”. During the last decade, several clinical studies have been conducted using heat as a therapeutic modality for cardiovascular diseases such as hypertension, arrhythmia, coronary artery disease and congestive heart failure (10). The findings in these clinical studies are congruent with epidemiological statistics showing that morbidity and mortality from cardiovascular etiologies are lower in hot environmental temperatures of various geographical regions (11,12,13,14).

Historically, thousands of years of practice in the use of hot water pools, sauna, Roman and Turkish baths, Japanese hot springs etc., have demonstrated that humans applying these methods have felt improved quality of life, relaxation and have general well being.

Thermal therapy (also referred to as an artificial fever) may generally be utilized with standard heart failure treatments to enhance clinical outcomes without additional risks for a large cross-section of heart failure patients. Importantly, thermal therapy has shown substantial clinical benefits across a variety of heart failure related areas such as hemodynamics, endothelial function, cardiac structure, arrhythmias and sudden death, among others. Given these important attributes and the potential clinical benefits, a large number of diseases may be helped with this therapy.

Small, safe increases in body core temperature (e.g., induced fever) have been shown to result in improvement in patients with various ailments including heart failure, hypertension, arrhythmia, stress and other ailments. The core temperature increase may be achieved by various means including exposure of whole and/or part of the body to a safe heat source (safe heat source levels such as radiation, electromagnetic energy, electrical stimulant and chemicals). Thermal therapy, such as an artificially induced fever, is a process that may help patients with many ailments.

The present disclosure relates to the field of treatment of ailments including, but not limited to, cardiovascular disease. More specifically, the present disclosure relates to methods and devices for treatment and/or prevention of cardiovascular disease, including arrhythmia, congestive heart failure and hypertension, as well as non-cardiovascular diseases.

In particular, what is disclosed is a method of stimulating, initiating, strengthening and enhancing the natural healing process of the body for dealing with various ailments at the molecular, cellular, organ, system and/or organism levels. This method may initiate and/or promote a natural, non-invasive body healing process at the molecular, cellular, tissue, organ and/or system levels. This method may provide the body the opportunity and environment to self heal. Using this method, cost of treatment may be drastically reduced in comparison to other methods or treatments of hypertension, heart failure, arrhythmia and other cardiovascular diseases. This method of treatment may be accessible to all those who need it without a need for hospitals, clinics, high health care costs, and may be applied anywhere. This method may be utilized and is beneficial to the young and the old. Thermal therapy may be applicable to acute, chronic disease, stress related ailments and/or pains.

The term “thermal therapy,” also referred to as artificial and/or induced fever, is used here to refer to any combination of factors such as higher temperature level, duration, frequency, and a method of delivering the heat, all of which may provide methods and means for delivery of heat from external sources and/or internal methods to increase core and/or surface body temperature to a safe level in order to initiate and activate therapeutic processes in the treatment of the various diseases, at the molecular, cellular, tissue, organ, system and/or organism levels, for example in arrhythmia, hypertension or congestive heart failure.

Thermal therapy utilizes relatively small increases in core body temperature intermittently for therapeutic purposes. This form of hyperthermia may be achieved by various means including the exposure of all or part of the patient's body to higher temperature environments for a short period of time, or by other means to raise the core body temperature. Turkish baths and Japanese hot springs are examples of methods which have been used for thousands of years (9) to increase body core temperature. Thermal underwear, heating pads, radiation, ultrasound, electromagnetic waves, high tech garments and other heating methods (e.g., drugs, chemicals) may also be used.

Thermal therapy is defined here as any means (e.g., dry heat, wet heat, sauna, infrared, microwave, ultrasound, thermal or other methods including electrical and electromagnetic waves as well as chemicals) that may cause increases to the body core and/or surface temperature and induce therapeutic effects. Thermal therapy may be applied in one session or multiple sessions, for various durations as required.

There may be a combination of factors such as higher temperature level, duration, frequency, or various other methods of increasing the temperature of a portion of the body or the whole body (e.g., core and/or surface) by external or internal methods.

A method for prevention and treatment of arrhythmias, heart failure and hypertension as well as other ailments is disclosed in which various pathways for reverse remodeling and vasodilatation improved signs and symptoms. Improved outcomes may include increases in nitric oxide and heat shock protein that enhance the body's healing power. The present disclosure may provide improved outcomes by exposure of all or part of the body to a safe temperature which is higher than normal body temperature, but lower than the endangering temperature, for a given range of frequencies and durations. The endangering temperature for the body or a portion of the body may be dependent on the patient and/or the portion of the body. Generally, an endangering temperature may be a temperature at which tissues and/or biological functions may be damaged and/or impaired. Raising the temperature by an unendangering amount and holding the raised temperature for an unendangering time period may prevent the risk of reaching the endangering temperature. These parameters may vary by patient and/or by the portion of the body to which the method is being applied.

Although the present disclosure includes description of theories and proposed mechanisms, the present disclosure is not bound to and is not reliant upon any of these theories or proposals.

### Treatment Regime

The present disclosure relates to methods and devices used to increase body temperature by various means for initiating, allowing, enhancing, stimulating, strengthening, helping and/or promoting the body's natural healing capacity of molecular, cellular, tissue and organ levels.

In general, thermal therapy may provide a way to treat and/or prevent an ailment in a patient. The body temperature of the patient is raised by a pre-determined amount. The increased body temperature is held for a pre-determined time period. These steps may be repeated until the desired result is achieved.

The pre-determined amount of temperature increase may be determined through experimentation, and may vary from patient to patient and/or for different ailments or desired results. For example, the body temperature may be raised by about 0.1° C. to about 2° C., typically so that the body temperature is above the patient's normal body temperature, but below temperatures that might endanger the patient. The temperature may be raised using a variety of methods known to those skilled in the art, and may involve exposing a portion or all of the patient's body to an increased temperature source.

Although some examples describe increasing the core body temperature of the patient, a desired therapeutic result may also be achieved by increasing the temperature of only a portion of the body. For example, there are specific beneficial proteins in the heart that local heating of the heart may release, without raising the core body temperature. There may also be specific applications in wound healing, where again the temperature is increased only for a portion of the body and the core body temperature is not raised.

The pre-determined time period may be determined through experimentation, and may vary from patient to patient and/or for different ailments or desired results. For example, the pre-determined time period may range from about 2 minutes to about 20 minutes.

Thermal therapy may be given to a patient repeatedly, for example daily for 1 to 24 weeks, depending on the desired result.

There are various means to increase body core temperature, which may be used in combination, including:

a. Heat sources

b. Chemical and/or drug induced

c. Radiation

e. Electrical

f. Electromagnetic

g. Ultrasound

h. Heat patches

i. Other means

An increase in core and/or surface body temperature may provide a means for the prevention and/or treatment of diseases including cardiovascular (e.g., hypertension, heart failure and arrhythmias) and non-cardiovascular ailments.

A sufficient degree of increased temperature may due to the combination of intensity, frequency, duration of treatment, patient conditions and other factors sufficient to initiate and activate the process to improve structural and functional factors including improvement in conditions at the cellular, molecular, tissue, organ and/or system levels.

In one example, one treatment regime increases the core body temperature to a level that is below the upper limits of safe temperature, without damaging the tissue. The core body temperature is increased by various degrees, for example up to 2° C. with sufficient time (in this example, up to 20 minutes exposure) and at a frequency of once or twice a day or as required for each individual and/or case. Alternate temperature ranges, frequencies and durations may also be used as long as local tissue temperature is below a tissue-damaging threshold, such as 43° C. This threshold may be dependent on case-by-case situations.

An effective degree of treatment may vary with the patient's condition, media of energy delivery and the stage of disease intensity. For example, it has been found that from about 0.1° C. up to about 2.0° C. may be a desirable amount to increase the body's temperature, with a combination of exposure time, duration, and heat intensity.

In one embodiment, one treatment regime may be the application of thermal therapy for a duration of about 2 minutes to about 20 minutes, up to a temperature of about 50° C., and at a frequency of about once a day, or periodically, for the duration of 2 weeks or until the patient's conditions improve. A combination of temperature, exposure time, duration, frequency of exposure and length of application are important and this disclosure stresses all of these combinations and permutations. A person skilled in the art would be able to vary and adjust the time duration, temperatures, frequency and length of the regime as suitable, depending on the patient, the ailment, and the desired result.

What is considered an effective amount of heat may vary with different factors. As an example, subjecting part of or the whole body to higher temperatures for a period of 10-20 minutes, every day for 1 week to several weeks, may initiate the process for improvement of conditions. In some cases, one treatment may be sufficient to initiate the processes or to obtain the desired result. Other treatment regimens may be possible, and the parameters may be varied to suit the patient, ailment and desired result.

### Methods of Increasing Body Temperature

Body temperature increase may be facilitated via various means including:

a. Chemical

b. Electromagnetic radiation

c. Radiant heat source

d. Heat exposure

e. Pharmaceutical agents

f. Ultrasound

g. Electrical energy

h. Other methods

Thermal sources may be safe and the device may be a thermal garment (e.g., a shirt or other clothes), heat belt, sauna (e.g., wet, dry or steam saunas), whirl pool, hot spring, thermal bed, ambient temperature and other methods such as various drugs, devices and other systems. Devices using ultrasound, electromagnetic fields, or other types of energy may also be used for thermal therapy. These devices and thermal sources may be used alone or in conjunction with each other to treat various ailments, including cancer, diabetes, obesity and mental disorders, among others.

A variety of thermal therapy methods including traditional sauna have been studied. One such method consists of warm water immersion of the patient's body in a semi-recumbent position at a water temperature of 41° C. (15). Another water-based method was reported by a German research group who investigated home-based hydrotherapy in patients with New York Heart Association (NYHA) Class II-III heart failure (16). In this form of hydrotherapy, patients receive warm peripheral baths to a maximum of 40° C. followed by the application of cold baths with a temperature less than 18° C., this process is repeated three times a day for six weeks. These studies resulted in clinically measurable improvements in patients with heart failure and excellent patient compliance (16). However, the main disadvantage of water immersion is the effects of hydrostatic pressure during water immersion which may lead to significant increases in right side intracardiac pressures. Instrumented studies have demonstrated that right atrial, pulmonary arterial, and pulmonary capillary wedge pressures increase significantly during warm water immersion, but not during dry sauna. The cause of increased rightsided pressures during water immersion may be related to an increase in venous return caused by hydrostatic pressure.

Another thermal therapy method studied was the dry sauna approach which utilizes infrared (IR) as a heat source. The absence of the hydrostatic component that occurs with water immersion makes this dry sauna method of treatment preferable, particularly in patients with severe congestive heart failure (17). This form of sauna has been studied extensively by several groups of Japanese researchers at the University of Kagoshima (8,17,18,19,20,21,22,23,). Those researchers utilized this approach by subjecting heart failure patients to dry sauna at a temperature of 60° C. for a period of 15 minutes per session. Other methods are currently being investigated, such as heat garments, chemicals and radiation.

Combination with Other Treatment

In one embodiment, the application of heat or thermal therapy may be combined with the administration of other treatments and management of the disease (for example, the introduction of drugs and/or devices). The combination may provide benefits both in terms of efficacy and/or safety. The thermal therapy technique may be used in conjunction with other management tools if the healthcare professional and patient prefer. In an embodiment, the thermal therapy may be applied to the patient in conjunction with drugs and/or devices that have been normally described and utilized for specific diseases. Existing drugs, devices and/or pharmaceutical agents for heart failure and hypertension may be delivered to the patient in combination with the application of thermal therapy.

Thermal therapy application may be combined with drugs such as beta adrenergic antagonists to increase the effectiveness of drugs and/or devices. This therapy may also be combined with other drugs such as Atenolol, Propranalol, Metoprolol and other agents.

Thermal therapy treatment may also be combined with the administration of a calcium channel blocker in order to increase efficiency of anti-arrhythmic therapy, hypertension and/or congestive heart failure. For example, calcium channel antagonist, Nifedipine, Verapamil or Diltiazem may be administered with thermal therapy.

Thermal therapy may be combined with anti-arrhythmic or anti-hypertension devices and/or congestive heart failure devices for the prevention or treatment of these disorders. Such devices as cardiac defibrillators and ventricular assist devices may be used with thermal therapy. Pacemakers and other devices and/or drugs may be used in combination with thermal therapy.

This disclosure describes possible pathways by which thermal therapy acts to treat cardiovascular and non-cardiovascular ailments. This disclosure is not limited by any theories or proposed mechanisms and/or pathways. Results of thermal therapy may be due to various pathways and processes from cellular and molecular to tissue, organ, system and/or organism levels.

Thermal therapy may activate biochemical and natural processes such as nitric oxide (NO) and heat shock proteins (HSPs) in order to achieve a healing response. Healing activities may also be achieved through other means such as decreasing oxidation and/or improving calcium ion movement.

Benefits of thermal therapy may include:

1. Reduction in intracardiac pressure.

2. Reduction of arrhythmia.

3. Reduction in cardiac dimension.

4. Reduction in brain natriuretic peptide (BNP).

5. Reduced ventricular dilation.

6. Increased nitric oxide (NO) bioavailability.

7. Increased heat shock protein (HSP) production.

8. Increase antioxidant enzyme production

9. Increased stroke volume.

10. Increased cardiac index.

11. Increased ejection fraction.

12. Increased physical capacity.

13. Reduction in anxiety, depression and stress.

14. Dilation of blood vessels.

15. Improved blood circulation.

16. Improved clinical outcomes of cardiovascular disease.

### Thermal Therapy for Treatment of Blood Pressure

Blood pressure and the incidence of heart failure and arrhythmia has been found to decrease, generally during warmer seasons and are higher in colder seasons in hypertensive patients in the same locations when other conditions are kept similar.

Increasing body core temperature has been found to reduce blood pressure, improving the condition of patients with arrhythmia and congestive heart failure and improving hemodynamics in patients. Improvements observed in hemodynamics, ejection fractions, reduction in arrhythmias and hypertension during and post thermal therapy support the hypothesis that body core temperature increase is a method of prevention and/or treatment of such disorders.

Body temperature may be increased by various methods including repeated, intermittent exposure to safe heat as required. In addition to treatment and/or prevention of ailments such as arrhythmias, heart failure and hypertension, thermal therapy may also reduce blood pressure, thus reducing afterload. It may increase vascular diameters, thus reducing afterload and ultimately helping patients with congestive heart failure, hypertension and arrhythmias.

### Thermal Therapy for Treatment of Renal Failure

One such example of an ailment where there is potential for the application of thermal therapy is in chronic renal failure or kidney disease. According to research, nitric oxide (NO) production and bioavailability is deficient in subjects with renal failure. Thermal therapy was found to increase the levels of nitric oxide in the body and activates other pathways, thereby acting to preserve renal function or slow down the progression of renal failure.

NO may be increased by thermal therapy, causing vasodilation and dampening of the norepinephrine effect. Thus, NO has many beneficial effects on overall health including, but not limited to:

a. Healthy immune system

b. Plays a positive role in healthy brain function

c. Has an antibacterial impact

d. Has antiviral properties

e. Helps oral health

f. Increases energy level

g. Improves concentration

h. Improves alertness and awareness

i. Improves self image

j. Impact on and increases alpha (α) and theta (θ) brain wave activities, thus improving mental health such as learning, memory and other functions of the cerebral cortex.

The mechanisms of nitric oxide generation and vasodilation outcome are proposed to be:

a. Increased temperatures cause various activities including action potential from the brain to vessels and heart.

b. This signal causes nitric oxide synthase (NOS)

c. The produced NO diffuse into the small muscle cells.

d. The NO binds with guanylyl cyclase causing cyclic GMP production (cGMP).

e. cGMP cause smooth muscle proteins to relax, thus the diameter and vessel is increased.

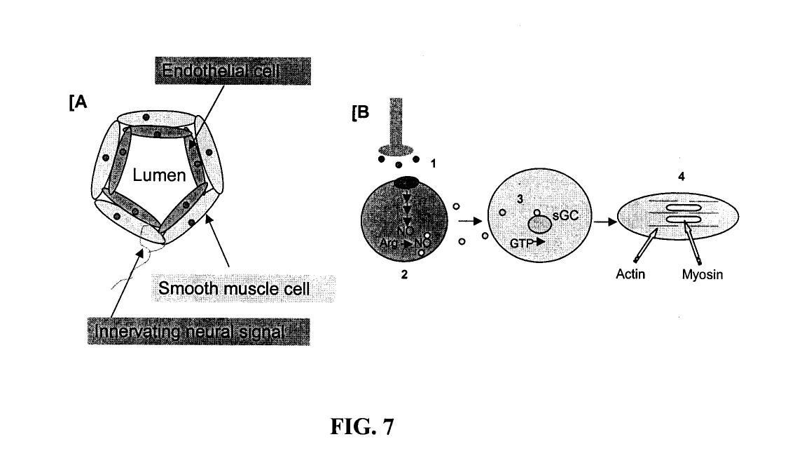

These mechanisms are also described in FIG. 7. FIG. 7 shows a schematic representation of Nitric Oxide (NO) production and a molecular pathway for vasodilation in a blood vessel. [A] Cross section of a blood vessel indicating the cell types and an incoming signal from a brain neuron caused by induced fever (Thermal Therapy). [B] A brief outline of the sequence of events involved in vasodilation induced by NO. 1) Axon releases acetylcholine (Ach) into the synaptic cleft 2) Ach or another hormone binds to an endothelial membrane protein inducing the production of NO with nitric oxide synthase (NOS) 3) NO diffuses to nearby cells (smooth muscle) and binds to soluble guanylyl cyclase promoting cyclic GMP production. 4) cGMP activates proteins are responsible for smooth muscle relaxation.

### Studies

Existing literature on the use of thermal therapy was retrieved by searching Medline and Embase databases. Relevant published papers (in English) from 1980 to December 2006 were examined. Studies were only included if the subjects suffered from heart failure and if assessments were made to quantitatively detect changes in heart failure parameters. All age groups and disease severity were included. Animal studies are mentioned only to support related clinical findings in human subjects. Statistically significant outcome measurements were considered and the results are grouped.

a. Improved endothelial function
increased NO production
increased HSP production
increased antioxidant enzyme production

b. Cardiac function:
increased ejection fraction
cardiac index
effective stroke volume

c. Decreased cardiac dimensions and dilation.

d. Decreased cardiothoracic ratio

e. Decreased cardiac arrhythmias

f. Decreased cardiac sudden death

g. Reduction in ventricular wall stretching

h. Reduction in level of brain natriuretic peptide (BNP)

i. Reduced edema

j. Reduced dyspnea (difficulty breathing)

k. Improvements in depression

l. Improvements in sleep disorders

m. Improved quality of life

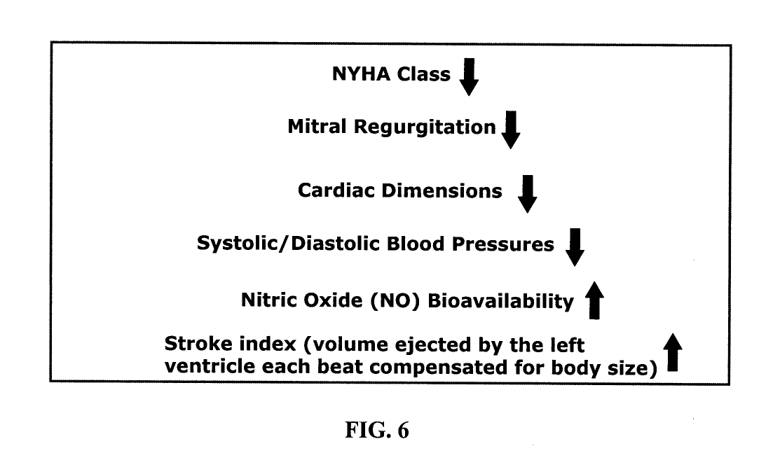

n. Other improvements as reflected in Table 1 and FIG. 6

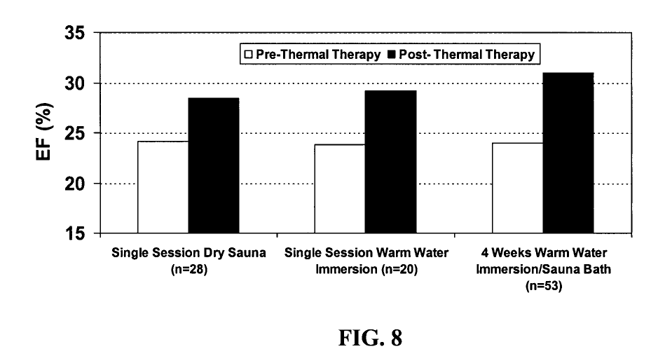

Thermal therapy studies have clinically examined the effect of both single and repeated thermal therapy treatments on a wide variety of patients with various stages and types of heart failure. FIG. 8 highlights the impact of various thermal therapy approaches on ejection fraction (EF) in NYHA Class II-IV congestive heart failure patients (18,20). Outcomes have been assessed by measuring the change in various disease indicators and related parameters such as ejection fraction (20), blood pressure (18), cardiac dimensions (8), cardiac arrhythmias (19), and quality of life (16). The documented outcomes and improvement in a wide variety of heart failure related parameters and markers are detailed in Table 1 and discussed in greater detail in the following section. The impact of thermal therapy on hypertension and arrhythmia has shown positive results as well.

The overall objective of the study and analysis was to assess the potential for hyperthermia (i.e., thermal therapy) as a treatment for many diseases. While focus has been placed on the main cardiovascular diseases (CVD) such as heart failure, arrhythmia and hypertension, this method may benefit other ailments as well.

The main focus was placed on heart failure, hypertension, and arrhythmia which are all key representatives of CVD. A majority of the relevant published papers (prior to December 2007) were included in the review and analysis. Studies specific to heart failure patients undergoing thermal therapy and that included measurement of heart failure related clinical parameters were quantitatively assessed.

Results: 1) Thermal therapy was found to have a positive impact on arrhythmias, hypertension, heart failure and other already defined CVD related parameters that were reported across multiple studies. 2) Significant improvements were noted across a wide scope of CVD related parameters in the areas of a) endothelial function, b) hemodynamics, c) cardiac structure, d) neurohormonal markers, and e) quality of life. 3) Of special note, thermal therapy also conveyed a strong anti-arrhythmic effect in heart failure patients, as noted in Table 1.

Conclusion: The clinical evidence highlights repeatable and compelling data that thermal therapy may provide an important and viable treatment of heart failure and other CVDs as well as non CVDs that are linked to heart failure.

Preponderance of Evidence: Most of the studies reviewed and analyzed have been:

a. Observational studies

b. Involve different forms of thermal therapy (dry heat, submerged, warm water, etc)

c. The preponderance of the evidence highlights repeatable and compelling data across a myriad of heart failure, hypertension and arrhythmia symptoms and markers that indicate significant clinical benefits which may be obtained using thermal therapy (Table 1).

d. No Adverse Events: Thermal therapy offers many potential benefits, since as a non-pharmacological treatment; it is generally devoid of adverse reactions with none of the studies reporting adverse events.

e. Widespread Applicability: Unlike exercise training, patients who are physically unfit, in frail health, aged, have severe heart failure, or orthopedic limitations would not generally be exempt from thermal therapy.

f. Quality of Life Improvements: Thermal therapy has also been shown to promote mental and physical relaxation leading to improved quality of life including increased appetite, sleep quality and general well being.

g. Applications: Application of this therapy may benefit many ailments, both cardiovascular and non-cardiovascular.

Studies have found that the risk of myocardial infarction (MI) and sudden death are lower during thermal therapy than other daily activities. Thermal therapy may decrease the rate of mitral regurgitation during and after thermal therapy. Repeated intermittent sessions of thermal therapy, every day for two weeks, have been shown to improve:

a. Dyspnea

b. Fatigue

c. Appetite

d. Sleeplessness

e. Edema

f. Constipation

g. Decrease New York Heart Association (NYHA) Classification

h. Maximize Exercise Capacity

i. Relaxation and Stress

j. Overall Quality of Life

k. Arrhythmia Reduction

l. Afterload

m. Vascular Resistance

Improvement in cardiac dimension, myocardial size, ejection fraction, cardiac index and neurohormonal response are only some of the benefits seen with thermal therapy.

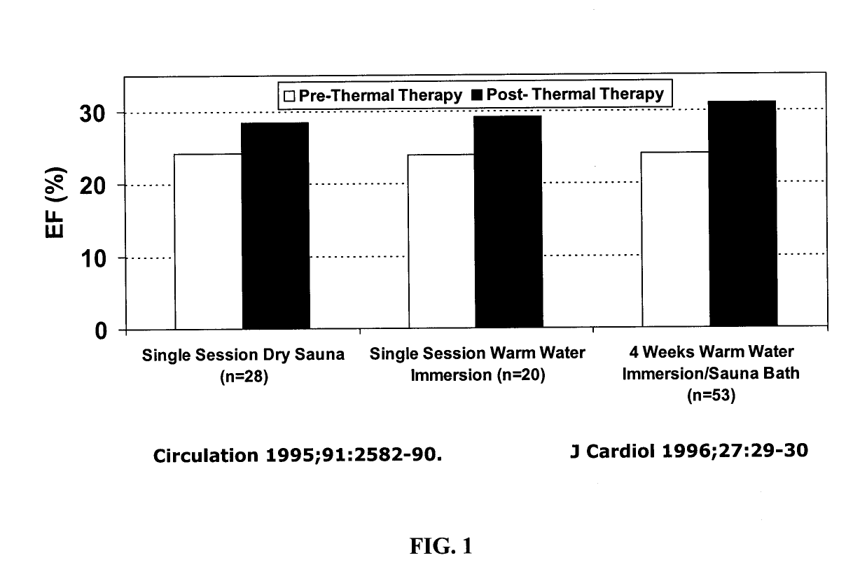

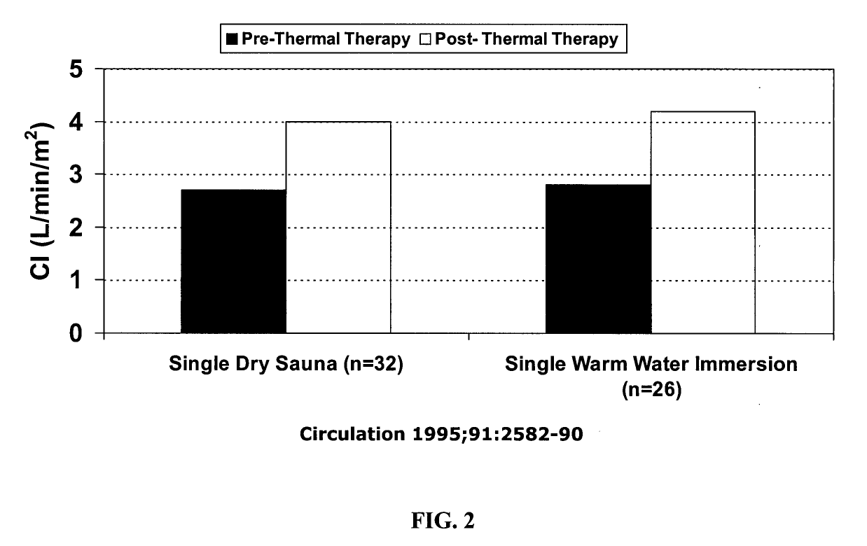

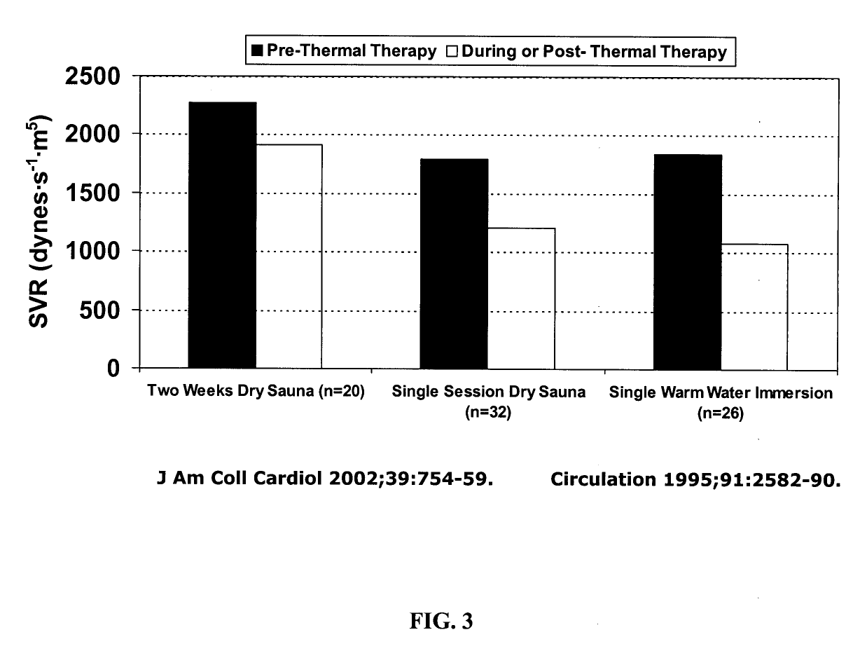

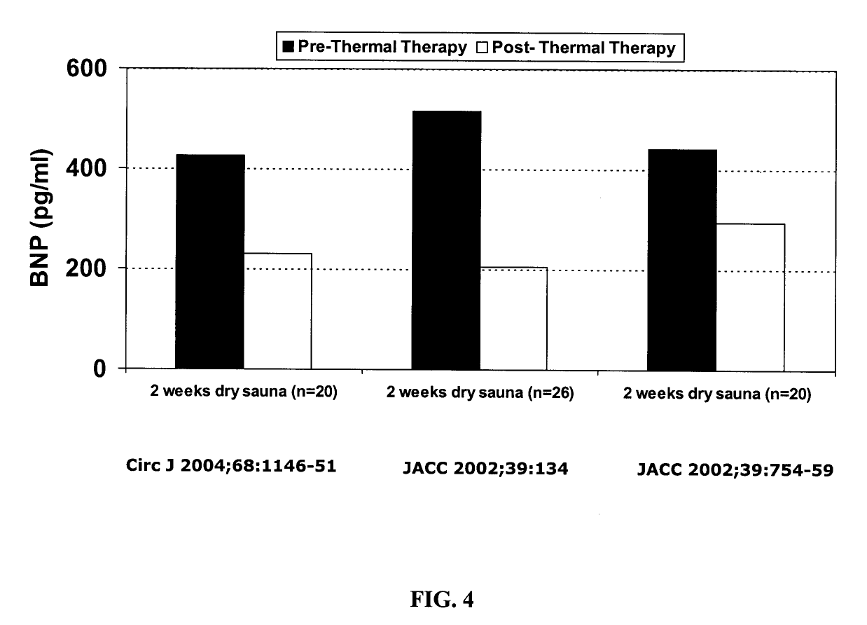

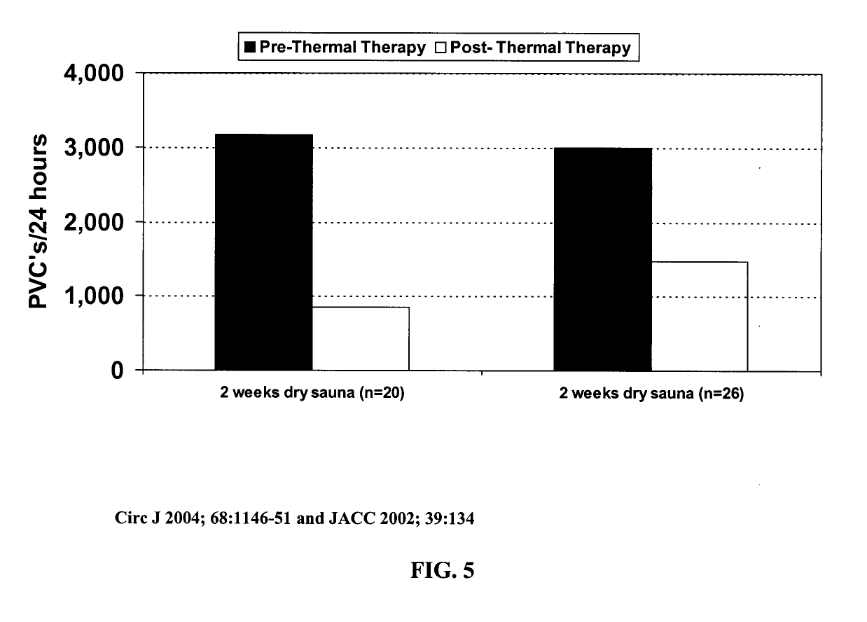

Based on data collected and analyzed, it has been shown that thermal therapy (also referred to as an artificially induced fever) has a positive impact on many ailments by improving various health indicators such as those illustrated in Table 1 and FIGS. 1 through 6.

The methods and devices disclosed here have been found to produce many improved outcomes including systolic and diastolic blood pressure reduction. Other outcomes may include reduction in vascular resistance which may help patients with hypertension, heart failure, arrhythmia, pulmonary hypertension, memory loss and other diseases.

The disclosure may apply to many ailments and those named here are examples and are intended only to show effectiveness in several diseases and should not be interpreted as limiting the scope and/or applications of this disclosure.

### Effect of Thermal Therapy on Endothelial Function

Endothelial dysfunction is a term used to describe the impaired ability of vascular endothelium to stimulate vasodilation (24). In heart failure, endothelium-dependant vasodilation is markedly reduced in response to acetylcholine (25) and reactive hyperemia (26). This eventually leads to increased vascular resistance (25,27,28,29). Animal studies have shown that thermal therapy may prevent the endothelial coronary dysfunction induced by ischemia reperfusion in rats (30,31). These studies have also demonstrated that thermal therapy leads to the induction of specific cardioprotective heat shock proteins (30).

In heart failure patients, the effect of thermal therapy on endothelial function was evaluated using several parameters either directly or indirectly. Several clinical studies have shown that systemic vascular resistance (SVR) decreases during and after thermal therapy sessions (8,18,22,23). The decrease in SVR ranged from 41.5% during thermal therapy (18) to 15.7% one day after a two-week treatment (8). This decrease in SVR during and after thermal therapy could be considered a potential sign of improved endothelial function in these patients.

A Japanese research group studied the effects of repeated thermal therapy on endothelial dysfunction using more specific parameters (8). Those researchers applied the dry sauna method for 2 weeks on patients with NYHA class II-III heart failure. Towards the end of the sessions, they evaluated endothelium-dependant vasodilation using the percent flow-mediated dilation (% FMD) method, as well as endothelium-independent vasodilation using sublingual nitrate administration (% NTG). The % FMD was found to be significantly increased after 2 weeks of sauna therapy by about 30%, but the % NTG was not. Additionally, systemic vascular resistance decreased and mean systolic blood pressure (SBP) dropped to 97 mm Hg, as measured one day after the two-week sauna treatment ended, compared with a baseline value of 107 mm Hg. The author hypothesized that the improvement in endothelial function after periodic thermal therapy may be caused by improved nitric oxide (NO) production which has already been clearly demonstrated in animal studies (32). Human studies in infants with severe heart failure also demonstrated increased urinary excretion of NO metabolites (e.g., nitrate and nitrite) following thermal therapy, suggesting increased NO activity as a result of thermal therapy (22).

### Effect of Thermal Therapy on NO and Nitric Oxide Synthase (NOS)

NO is a gaseous free radical molecule that is released from NOS in endothelial cells and it diffuses to the neighboring smooth muscle cell where it activates soluble guanylyl cyclase (sGC), the enzyme that forms the secondary messenger cyclic guanosine-3,5-monophosphate (cGMP) which in turn activates cGMP-dependent protein kinase (61). Once cGMP is generated in the smooth muscle cells, it acts as a messaging molecule that induces the action of myosin light chain phosphatase (MLCP) which is an enzyme that dephosphorylates r-MLC resulting in smooth muscle relaxation (62). Thus, the production of cGMP (in a muscle cell) due to NO (produced in an endothelial cell) is critical to the activation of MLCP which induces smooth muscle relaxation and the subsequent vasodilation of blood vessels.

It is believed that NO acts to fine tune and optimize cardiac pumping function (63). Extensive animal studies have produced results demonstrating that NO is an important mediator in coping with the hemodynamic deterioration in CHF (64-67). In response to an increase in metabolic demand (64), NO was found to contribute to the preservation of coronary blood flow (CBF) in dogs with HF (65). Furthermore, in pigs, the inhibition of NO biosynthesis in the acute phase of CHF was found to induce a significant increase in peripheral arterial resistance and arterial elastance (calculated as end diastolic pressure divided by stroke volume) which was accompanied by a reduction in cardiac output (66). These results show the importance of NO in maintaining proper hemodynamic status in CHF.

Similar studies of endomyocardial biopsies in humans have shown that patients with a preserved ability to produce NO in cardiac tissue have a better hemodynamic profile (68). This was evidenced by a less restrictive filling pattern, more compliant left ventricles, meaning they are more likely to stretch, and increased cardiac work, which is a greater volume of blood ejected for a specified amount of work (68). The impairment of endothelium-dependant production of NO is a primary contributor to the elevated peripheral vascular pressure, and thus contributes to deteriorating clinical symptoms (69).

The use of thermal therapy has shown increases in NO production. It has been found that thermal treatment of infants with CHF was accompanied by increased metabolites of NO (i.e. nitrites and nitrates) in urine which suggests a rise in NO (70). Some studies have also shown that thermal treatment improved endothelium-dependent vasodilation (71,72), which is a sign of a heat-induced increase in NO production from vascular endothelium (71).

One mechanism that has been proposed for the increase NO production is the upregulation of the enzyme that produces NO, namely NOS. The incubation of bovine endothelial cell culture at 42° C. resulted in significantly increased eNOS activity followed by and NO release twenty-four hours later (73). NOS was also found to increase after a single heat exposure in the aortic endothelium of incubated rats (73). In another study, golden hamsters were treated daily with infrared sauna at 39° C. for 15 min followed by 30° C. for 20 min for 4 weeks (74). Ikeda et al found that the sauna treated hamsters had significantly (50%) increased eNOS expression in their aortas compared to hamsters that were not thermally treated (75). The repeated sauna treatment also increased the aortic NOS expression in cardiomyopathic animals, after it was downregulated as a consequence of heart failure (75).

There are studies that provide direct evidence that repeated thermal therapy upregulates NOS (73,74,75). The upregulation of NOS after thermal treatment was found to increase the bioavailability of NO, and subsequently, improve endothelial dysfunction (75) which would result in decreased vasoconstriction (73) and improved cardiac function.

### Effect of Thermal Therapy on Heat Shock Proteins (HSPs)

Cells have the ability to of defend themselves from various stressors by activating a genetic program with the production of substances known as heat shock proteins (HSPs) (76). HSPs act as molecular chaperones by maintaining proper protein assembly, folding and transport (77). HSPs are named as such because they are activated by heat, which was the first discovered trigger for enhanced transcription of the proteins (76). Subsequently, these proteins have also been shown to increase in the presence of other stressful stimuli, including ischemia, hypoxia, oxidative injury, ethanol, heavy metals, and endotoxemia (78). A physiological stressor (i.e. heat) stimulates HSPs to break lose of heat shock factors (HSFs) which are phosphorylated and translocated to the nucleus where they bind to DNA in order to induce the transcription of more HSP mRNA (79).

HSPs that are most often associated with cardiovascular disease are Hsp27, Hsp60, Hsp65, Hsp70, and Hsp90 (76). Several studies where HF was induced in animals by aortic ligation have shown a transient over expression of Hsp70 during the first stages of HF (80-82). Furthermore, evidence shows the ability of hearts to produce Hsp27, (81) Hsp72, (81,83) and Hsp73 (83) 8 weeks after the induction of CHF.

Whole body heating in rats was associated with the improvement of cardiac functional recovery after a global ischemic insult and was associated with an increase in Hsp70 and antioxidant enzymes (84). Following an ischemia-reperfusion insult, the induction of the Hsp70 family by thermal exposure was associated with an accelerated recovery of cardiac contractile function (85). Protection against reperfusion arrhythmias was found to be associated with a two-fold increase in endogenous catalase activity and expression of the inducible heat shock (stress) protein Hsp70 (86). Given that Hsp27, Hsp70 and Hsp90 were reported to protect cardiomyocytes against apoptosis (87,88), it stands to reason that HSP could be a strong support for the failing heart.

### Effect of Thermal Therapy on Oxygen Free Radicals and Antioxidant Enzymes

Oxygen free radicals are chemical substances produced by the reduction of oxygen during many cellular reactions (89). A free radical is an extremely unstable and reactive molecule that rapidly enters into reaction with nearby molecules (90). There are three types of oxygen derived free radicals. One is superoxide anion (O²⁻.), which is the most toxic of the three types. The antioxidant enzyme superoxide dismutase (SOD) converts superoxide anion to another free radical, which is hydrogen peroxide (H₂O₂) and it is broken down to water and oxygen with the antioxidants catalase and glutathione peroxidase (GPx). The third free radical is hydroxyl radical (OH) which is highly toxic and it is produced by the reaction of superoxide with hydrogen peroxide or hydrogen peroxide with a metal ion (89). SOD, catalase, as well as (GPx) are the three main antioxidant enzymes that are imperative to the human body's self defense (91) and can be outlined by the following reaction scheme:

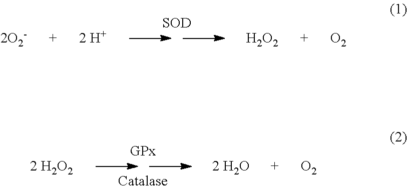

At the organ level, free radicals can damage the cardiac muscle by multiple mechanisms such as: cell membrane oxidation, protein denaturation and DNA mutation. Generally, they involve direct toxicity by inducing both necrosis and apoptosis, which impairs myocardial function and induces cardiac arrhythmias (92). Findings from in vivo studies show a reduction in the antioxidant enzyme activities of SOD, GPx, and catalase in animals with CHF (93,94). The reduction was found to be associated with HF manifestations such as increased left ventricular end diastolic (LVED) pressure, dyspnea, and ascites (93), and a progressive rise in oxidative stress markers (94). Similarly, studies with human CHF patients reflected declined SOD content in erythrocytes (95), myocardium (93) and endothelium-bound SOD (96). Collectively, this data supports the concept that oxidative stress may be caused by antioxidant enzyme deficit in patients with cardiovascular ailments.

Several studies have shown the ability of whole body hyperthermia to have a protective effect against oxidative stress. Mammalian cells can respond to sudden heat rise by up-regulating the mRNAs of several antioxidant enzymes namely, catalase and SOD (97).

Catalase activity, a major indicator of antioxidant enzyme activity was repeatedly found to be significantly increased along with a parallel decrease in the production of free radicals in the heart of rats (98-102) and rabbits (103,104). The other important antioxidant enzyme, SOD, was found to be enhanced by hyperthermia (105,106). LV biopsies from pig hearts infused with warm blood at 42° C. had a significant increase in SOD activity (105). Therefore heat can be a powerful inducer of antioxidant enzyme synthesis which will destroy the dangerous oxygen radicals.

### Effect of Thermal Therapy on Cardiac Pumping Function

Cardiac pumping function was assessed in several studies by measuring ejection fraction, cardiac index and stroke index. In a trial using a single dry sauna treatment, it was shown that cardiac index and stroke index increased by 48.1% and 13.9% respectively in NYHA class II-IV patients during treatment (18). In addition, ejection fraction was increased from 24.1% to 28.5% during thermal therapy in this study. Subsequent studies utilizing repeated thermal therapy treatments (1-2 times/day, 5 times/week for 4 weeks,) resulted in increasing the ejection fraction of NYHA Class III and IV patients from 24% to 31% (20). These patients also averaged more than 1 Class improvement in the NYHA scale after 4 weeks of repeated thermal therapy treatment.

### Effect of Thermal Therapy on Cardiac Dimensions

The chronic increase in pressure load due to heart failure results in dilation of the cardiac chambers and hypertrophy of the myocardium (33). The level of the resultant cardiomegaly is an indicator of the severity of the disease. Thermal therapy was found to positively influence cardiac dimensions. Cardiothoracic ratio (CTR) was shown to decrease after repeated applications of thermal therapy (8,19,20,34). Two weeks of dry thermal therapy resulted in decreased CTR from 60% to 57% in patients with both dilated and ischemic cardiomyopathy (34). In another study, four weeks of thermal therapy resulted in a reduction of CTR from 61% to 55% in NYHA class III-IV patients (20). The echocardiography parameter Left Ventricular End Diastolic Dimension (LVEDD) has also shown a similar trend to the reductions in CTR. LVEDD was shown to decrease by 3.4% and 6.1% after 2 and 4 weeks of dry thermal therapy respectively (8,20). These decreases in cardiac dimension provide an indicator of decreased pressure load and improved overall hemodynamic status in patients treated with thermal therapy.

### Effect of Thermal Therapy on Cardiac Arrhythmias & Sudden Death

Patients with heart failure have a high prevalence of potentially serious arrhythmias and, consequently, a high incidence of sudden cardiac death (35,36,37). The presence of ventricular arrhythmias also defines a high-risk patient group with either ischemic or non-ischemic cardiomyopathy (38,39,40,41). This gives special importance to the effect of thermal therapy on cardiac arrhythmias.

While the potential for increased arrhythmias during thermal therapy was initially a major concern with sauna therapy, early observations in post-myocardial infarction (MI) patients found they tolerated thermal therapy without increased risk for cardiac arrhythmias and other complications (42). In that study, sauna bathing at 90° C. did not increase the risk of arrhythmias to levels higher than those of other everyday stress situations. Subsequently, the effects of a 60° C. dry sauna on arrhythmias was examined specifically in patients with advanced heart failure and resulted in significant reductions in cardiac arrhythmia (34). This study, in 26 patients with dilated cardiomyopathy and ischemic cardiomyopathy showed a significant decrease in premature ventricular contractions (PVC's) from a mean of 2,993 to 1,476 per 24 hour period following 2 weeks of thermal therapy. An additional study with 30 NYHA Class II-III patients reported a 73% reduction in PVC's (from 3161 to 848 per 24 hour period) following 2 weeks of thermal therapy (19). Other forms of arrhythmias, such as couplets and episodes of ventricular tachycardia, were also found to be decreased by 50% and 75% respectively.

In addition to arrhythmia reduction, heart rate variability (HRV) was also found to be significantly increased following thermal therapy (19). Increased HRV is a sign of improved cardiac rhythm (43,44), while a low level of HRV is associated with an enhanced risk of ventricular fibrillation and an overall poor prognosis for heart failure patients (45). Based on various studies, it has been speculated that the decreased incidence of ventricular arrhythmias following thermal therapy may be attributed to decreased ventricular wall stretching (19).

Effect of Thermal Therapy on Brain Natriuretic Peptide and other Neurohormonal Markers

There is evidence (50) of short-term hormonal changes during sauna bathing, for example, in norepinephrine (2- to 4-fold increase), renin activity (1.5- to 2-fold increase), angiotensin II (3-fold increase), and aldosterone (3- to 6-fold increase). In terms of longer-term impact, a 2-week study utilizing dry thermal therapy found no sustained changes in epinephrine, norepinephrine, or dopamine levels; however, significant reductions in brain natriuretic peptide (BNP) from baseline were noted. (8,19)

The level of brain natriuretic peptide (BNP) is an important prognostic indicator in patients with heart failure (46,47). The mean value of BNP was found to drop significantly in heart failure patients who received dry thermal therapy for 2 weeks (8,19,34). One study showed a drop from 514 to 204 pg/ml (a 60.3% decrease) (34). Another study showed a significant correlation between the percent improvement in BNP concentration and the change in % FMD; with increased % FMD being a potential indicator of improved endothelial function in these patients (8).

### Effect of Thermal Therapy on Quality of Life & Heart Failure Related Symptoms

Heart failure is a debilitating disease; patients usually suffer from low exercise capacity in addition to many restrictive symptoms such as leg edema and dyspnea. Besides the physical manifestations, depression and sleep disorders are not infrequent in such patients. Consequently, the quality of life (QOL) deteriorates with the progression of heart failure. The impact of thermal therapy on the QOL of heart failure patients has been the subject of various studies.

A German study on home-based hydrotherapy conducted QOL studies during a 6 week course of thermal therapy in NYHA II-III patients (16). The QOL, which was assessed using a validated questionnaire, was significantly improved in terms of physical capacity, enjoyment and relaxation. Heart failure related symptoms were also found to be significantly improved in this study.

A group of Swedish researchers examined an exercise program in a temperature-controlled swimming pool (48). In this study, 15 patients with NYHA class II-III heart failure were instructed to perform exercises in the warm water (30-34° C.) for 45 minutes, 3 times a week for 8 weeks. The patients who exercised in warm water saw significant improvement in terms of maximum exercise capacity. Two different questionnaires, the Short Form-36 Health Survey Questionnaire and the Minnesota Living with Heart Failure Questionnaire were used. The results demonstrated that exercise improvement was also associated with improvement in the QOL for these patients.

Dry sauna thermal therapy was also found to improve other related symptoms in heart failure. One thermal therapy study noted significantly decreased rates of mitral regurgitation (MR) during and after thermal therapy in 20 of 26 patients with MR at baseline (18). Two weeks of dry thermal therapy in 20 patients with dilated and ischemic cardiomyopathy was found to improve dyspnea, fatigue, edema, sleeplessness, appetite loss, and constipation in 17 out of 20 patients (19). Longer treatments for 4 weeks were also shown to significantly decrease NYHA class (1.2 class reduction) in a study involving 56 NYHA class patients (20).

### Safety of Thermal Therapy

When considering the potential use of thermal therapy as a therapeutic modality, one may question the safety of this practice. Since heart failure is a complex syndrome that is frequently compounded by other conditions such as coronary artery disease (CAD) and myocardial infarction (MI) history, it is of paramount importance to understand the established safety profile of thermal treatment therapy.

A great deal of experience of thermal exposure with conditions such as CAD came from the studies on Finnish sauna (49). The benefits and risks of Finnish sauna were studied comprehensively in a meta-analysis that covered literature from 1966 to 2000 (50). This meta-analysis included 130 original papers that examined sauna treatment in relation to the major health problems and physiological systems. The authors reported that sauna treatment is safe for most individuals with coronary heart disease, stable angina pectoris and those with old MI (50). Patients with coronary heart disease were found to tolerate saunas well; only 2% reported chest pain during the sauna treatment whereas 60% had chest pain during normal daily life (50). Other reports from Norway and Germany supported that a sauna is well tolerated by CAD patients (49). When comparing thermal treatment with exercise, several studies reported that myocardial ischemia in CAD during saunas were found to be significantly less than during exercise (51,52).

It was found that the risk of myocardial infarction (53) and sudden death (54) are lower during sauna bathing than during other daily activities. A 10-year follow-up of 117 post-MI patients showed that 82% continued regular sauna use (53). During the 10-year follow-up, no occurrences of sudden death, re-infarction, or serious arrhythmia were attributed to sauna bathing in post-MI patients. In addition, cardiac arrhythmias were rare during thermal therapy compared with during physical exercise.

The ability of patients to maintain standard heart failure medication use when receiving thermal therapy is vitally important. Reports from Finnish sauna studies have shown that the combination of sauna with cardiovascular drugs, such as calcium antagonist, digitalis, diuretics, and long-acting nitrates, does not produce harmful reactions (49). One early report suggested Beta-blockers, particularly those with a marked bradycardic effect, may impact the increase in heart rate due to heat stress thus creating a hypotensive reaction. However in more contemporary studies, patients who received thermal therapy continued using their regular medication for heart failure before and during the course of thermal therapy without reported adverse effects (8,20). The effect of hyperthermia on the absorption, distribution, and elimination of orally administered drugs, including propranolol, captopril, and midazolam is minor (50). However, the absorption of transdermally and subcutaneously administered drugs was found to be increased which should be taken into consideration during the application of thermal therapy.

### Cautions and Contraindication of Thermal Therapy

There are few conditions that were found to be negatively affected by hyperthermic exposure, or are a contraindication for this practice. One group at risk could be patients on the antiepileptic drug topiramate. Environmental temperature was found to be a risk factor for topiramate-related hyperthermia (55); therefore, thermal therapy is not recommended for patients undergoing treatment with topiramate. Patients with pulmonary hypertension should also be cautioned. While no specific mention of pulmonary hypertension was found in the literature reviewed, the monograph for epoprostenol sodium, a vasodilator used in the treatment of primary pulmonary hypertension, indicates that patients should avoid situations that promote vasodilation such as saunas, hot baths, and sunbathing. Severe hypotension has been seen in patients treated with chronic epoprostenol infusions under such circumstances. Another risk factor could be pregnancy. Although there is evidence that sauna bathing is safe during pregnancy (50), sauna bathing for pregnant women in early pregnancy was suspected to increase the risk for neural tube defects (56,57).

Other conditions that were reported to be contraindicated for thermal therapy include:

Unstable angina pectoris, i.e. angina that has recently developed for the first time, or if pre-existing angina has worsened for no apparent reason (58).

Recent myocardial infarction (50). However, in a study that involved 102 MI patients, the mean time for re-starting sauna bathing for people accustomed to Finnish sauna was reported to be 6.7 weeks (the range was 3 to 24 weeks) (42).

Decompensated heart failure, uncontrolled hypertension and/or severe aortic stenosis (9).

### Advantages of Thermal Therapy

What has been disclosed is a method for thermal therapy, which is a non-invasive, non-drug therapy. The increased body (core and surface) temperature may be superior to the conventional methods for reasons which may include:

a. it is non-invasive

b. does not have side impacts (no chemical uptake)

c. enhances and initiates healing process at the molecular and cellular levels

d. increases vasodilatation by NO production, causing a reduction in blood pressure and afterload

e. activates heat shock protein processes

f. activates natural antioxidant enzyme production

g. relaxing

h. no pain is involved

i. initiates, enhances, strengthens and stimulates natural body healing process

Thermal therapy may generally be utilized with practiced heart failure treatments to enhance clinical outcomes without additional risks for a large cross-section of heart failure patients. Importantly, thermal therapy has shown substantial clinical benefits across a variety of heart failure related areas such as hemodynamics, endothelial function, cardiac structure, arrhythmias and sudden death, among others. Given these attributes and the potential clinical benefits, a large number of diseases may be helped with this therapy. All examples and embodiments are provided for the purpose of illustration only and are not intended to be limiting. All references and documents mentioned are hereby incorporated by reference in their entirety.

### REFERENCES

1. Haldeman G A, Croft J B, Giles W H, Rashidee A. Hospitalization of patients with heart failure: National Hospital Discharge Survey, 1985 to 1995. Am Heart J 1999;137:352-60.

2. Crawford M H, Dimarco J P, Paulus J W. Heart failure and cardiomyopathy: Special problems in chronic heart failure: Adjunctive therapies for management of heart failure, Cardiology, 1st ed. London: Mosby; 2003. p. 9.5-9.6.

3. Stewart S, MacIntyre K, Hole D, Capewell S, McMurray J. More ‘malignant’ than cancer? Five-year survival following a first admission for heart failure. Eur J Heart Fail 2001;3:315-22.

4. Wilson E. Congestive heart failure: A national priority. Can. J Cardiol. 2001;17:1243-44.

5. American Heart Association. Heart Disease and Stroke Statistics—2007 Update. 2007. Dallas, Tex., American Heart Association. Ref Type: Report

6. Strategic priorities of the WHO Cardiovascular Disease program. On the Internet at 2007;http://www.who.int/cardiovascular_diseases/priorities/en/.

7. Weber A A, Silver M A. Heat therapy in the management of heart failure. CHF 2007;13:81-83.

8. Kihara T, Biro S, Imamura M, Yoshifuku S, Takasaki K, Ikeda Y et al. Repeated sauna treatment improves vascular endothelial and cardiac function in patients with chronic heart failure. J Am Coll Cardiol 2002;39:754-59.

9. Keast M, Adamo K. The Finnish sauna bath and its use in patients with cardiovascular disease. J Cardiopulm Rehabil 2000;20:225-30.

10. Nguyen Y, Naseer N, Frishman W. Sauna as a therapeutic option for cardiovascular disease. Cardiol Rev 2004;12:321-24.

11. Nayha S, Hassi J. Cold and mortality from ischaemic heart disease in northern Finland. Arctic Med Res 1995;54:19-25.

12. Katz A, Biron A, Ovsyshcher E, Porath A. Seasonal variation in sudden death in the Negev desert region of Israel. Isr Med Assoc J 2000;2:17-21.

13. Hassi J, Remes J, Kotaniemi J T, Kettunen P, Nayha S. Dependence of cold-related coronary and respiratory symptoms on age and exposure to cold. Int J Circumpolar Health 2000;59:210-15.

14. Isezuo S A. Seasonal variation in hospitalisation for hypertension-related morbidities in Sokoto, north-western Nigeria. Int J Circumpolar Health 2003;62:397-409.

15. Tei C, Horikiri Y, Park J C, Jeong J W, Chang K S, Tanaka N et al. Effects of hot water bath or sauna on patients with congestive heart failure: Acute hemodynamic improvement by thermal vasodilation. J Cardiol 1994;24:175-83.

16. Michalsen A, Ludtke R, Buhring M, Spahn G, Langhorst J, Dobos G J. Thermal hydrotherapy improves quality of life and hemodynamic function in patients with chronic heart failure. Am Heart J 2003;146:E11.

17. Tei C, Tanaka N. Comprehensive therapy for congestive heart failure: a novel approach incorporating thermal vasodilation. Intern Med 1996;35:67-69.

18. Tei C, Horikiri Y, Park J C, Jeong J W, Chang K S, Toyama Y et al. Acute hemodynamic improvement by thermal vasodilation in congestive heart failure. Circulation 1995;91:2582-90.

19. Kihara T, Biro S, Ikeda Y, Fukudome T, Shinsato T, Masuda A et al. Effects of repeated sauna treatment on ventricular arrhythmias in patients with chronic heart failure. Circ J 2004;68:1146-51.

20. Tei C, Tanaka N. Thermal vasodilation as a treatment of congestive heart failure: A novel approach. J Cardiol 1996;27:29-30.

21. Ikeda Y, Biro S, Kamogawa Y, Yoshifuku S, Kihara T, Minagoe S et al. Effect of repeated sauna therapy on survival in TO-2 cardiomyopathic hamsters with heart failure. Am Heart J 2002;90:343-45.

22. Sugahara Y, Ishii M, Muta H, Egami K, Akagi T, Matsuishi T. Efficacy and safety of thermal vasodilation therapy by sauna in infants with severe congestive heart failure secondary to ventricular septal defect. Am Heart J2003;92:109-13.

23. Tei C. Thermal therapy for congestive heart failure: Estimation by TEI index. J Cardiol 2001;37:155-59.

24. Kurowska E M. Nitric oxide therapies in vascular diseases. Curr Pharm Des 2002;8:155-66.

25. Kaiser L, Spickard R C, Olivier N B. Heart failure depresses endothelium-dependent responses in canine femoral artery. Am J Physiol 1989;256:H962-H967.

26. Drexler H, Hornig B. Endothelial dysfunction in human disease. J Mol Cell Cardiol 1999;31:51-60.

27. Kubo S H, Rector T S, Bank A J, Williams R E, Heifetz S M. Endothelium-dependent vasodilation is attenuated in patients with heart failure. Circulation 1991;84:1589-96.

28. Katz S D, Biasucci L, Sabba C, Strom J A, Jondeau G, Galvao M et al. Impaired endothelium-mediated vasodilation in the peripheral vasculature of patients with congestive heart failure. J Am Coll Cardiol 1992;19:918-25.

29. Drexler H. Endothelium as a therapeutic target in heart failure. Circulation 1998;98:2652-55.

30. Amrani M, Corbett J, Allen N J, O′Shea J, Boateng S Y, May A J et al. Induction of heat-shock proteins enhances myocardial and endothelial functional recovery after prolonged cardioplegic arrest. Ann Thorac Surg 1994;57:157-60.

31. Joyeux M, Bouchard J F, Lamontagne D, Godin-Ribuot D, Ribuot C. Heat stress-induced protection of endothelial function against ischaemic injury is abolished by ATP-sensitive potassium channel blockade in the isolated rat heart. Br J Pharmacol 2000;130:345-50.

32. Harris M B, Blackstone M A, Ju H, Venema V J, Venema R C. Heat-induced increases in endothelial NO synthase expression and activity and endothelial NO release. Am J Physiol Heart Circ Physiol 2003;285:H333-H340.

33. Julian D G, Cowan J C. Cardiology, 6 ed. London: Bailliere Tindall; 1992.

34. Kihara T, Biro S, Imamura M, Yoshifuku S, Takasaki K, Otsuji Y et al. Effects of sauna therapy on cardiac arrhythmia in patients with chronic heart failure. J Am Coll Card 2002;39:134.

35. Meinertz T, Hofmann T, Kasper W, Treese N, Bechtold H, Stienen U et al. Significance of ventricular arrhythmias in idiopathic dilated cardiomyopathy. Am J Cardiol 1984;53:902-07.

36. Maskin C S, Siskind S J, LeJemtel T H. High prevalence of nonsustained ventricular tachycardia in severe congestive heart failure. Am Heart J1984;107:896-901.

37. Koseki Y, Watanabe J, Shinozaki T, Sakuma M, Komaru T, Fukuchi M et al. Characteristics and 1-year prognosis of medically treated patients with chronic heart failure in Japan. Circ J2003;67:431-36.

38. Holmes J, Kubo S H, Cody R J, Kligfield P. Arrhythmias in ischemic and nonischemic dilated cardiomyopathy: Prediction of mortality by ambulatory electrocardiography. Am J Cardiol 1985;55:146-51.

39. Dargie H J, Cleland J G, Leckie B J, Inglis C G, East B W, Ford I. Relation of arrhythmias and electrolyte abnormalities to survival in patients with severe chronic heart failure. Circulation 1987;75:IV98-107.

40. Stevenson W G, Sweeney M O. Arrhythmias and sudden death in heart failure. Jpn Circ J 1997;61:727-40.

41. Doval H C, Nul D R, Grancelli H O, Varini S D, Soifer S, Corrado G et al. Nonsustained ventricular tachycardia in severe heart failure: Independent marker of increased mortality due to sudden death. GESICA-GEMA Investigators. Circulation 1996;94:3198-203.

42. Luurila O J. Arrhythmias and other cardiovascular responses during Finnish sauna and exercise testing in healthy men and post-myocardial infarction patients. Acta Med Scand Suppl 1980;641:1-60.

43. Bilchick K C, Fetics B, Djoukeng R, Fisher S G, Fletcher R D, Singh S N et al. Prognostic value of heart rate variability in chronic congestive heart failure (Veterans Affairs' Survival Trial of Antiarrhythmic Therapy in Congestive Heart Failure). Am J Cardiol 2002;90:24-28.

44. Fauchier L, Babuty D, Cosnay P, Fauchier J P. Prognostic value of heart rate variability for sudden death and major arrhythmic events in patients with idiopathic dilated cardiomyopathy. J Am Coll Cardiol 1999;33:1203-07.

45. Koyama J, Watanabe J, Yamada A, Koseki Y, Konno Y, Toda S et al. Evaluation of heart-rate turbulence as a new prognostic marker in patients with chronic heart failure. Circ J 2002;66:902-07.

46. Troughton R W, Frampton C M, Yandle T G, Espiner E A, Nicholls M G, Richards A M. Treatment of heart failure guided by plasma aminoterminal brain natriuretic peptide (N-BNP) concentrations. Lancet 2000;355:1126-30.

47. Yasue H, Yoshimura M, Sumida H, Kikuta K, Kugiyama K, Jougasaki M et al. Localization and mechanism of secretion of B-type natriuretic peptide in comparison with those of A-type natriuretic peptide in normal subjects and patients with heart failure. Circulation 1994;90:195-203.

48. Cider A, Schaufelberger M, Sunnerhagen K S, Andersson B. Hydrotherapy—a new approach to improve function in the older patient with chronic heart failure. Eur J Heart Fail 2003;5:527-35.

49. Luurila O J. The sauna and the heart. J Intern Med 1992;231:319-20.

50. Hannuksela M L, Ellahham S. Benefits and risks of sauna bathing. Am J Med 2001;110:118-26.

51. Giannetti N, Juneau M, Arsenault A, Behr M A, Gregoire J, Tessier M et al. Sauna-induced myocardial ischemia in patients with coronary artery disease. Am J Med 1999;107:228-33.

52. Hoffmann A, Muller M, Niederhauser H U. Are swimming and sauna hazardous in the rehabilitation of heart patients? English abstract. Schweiz Med Wochenschr 1983;113:1054-57.

53. Eisalo A, Luurila O J. The Finnish sauna and cardiovascular diseases. Ann Clin Res 1988;20:267-70.

54. Luurila O J. Cardiac arrhythmias, sudden death and the Finnish sauna bath. Adv Cardiol 1978;25:73-81.

55. Ziad E K, Rahi A C, Hamdan S A, Mikati M A. Age, dose, and environmental temperature are risk factors for topiramate-related hyperthermia. Neurology. 2005;65:1139-40.

56. Moretti M E, Bar-Oz B, Fried S, Koren G. Maternal hyperthermia and the risk for neural tube defects in offspring: Systematic review and meta-analysis. Epidemiology. 2005;16:216-19.

57. Waldenstrom U. Warm tub bath and sauna in early pregnancy: Risk of malformation uncertain. Acta Obstet Gynecol Scand 1994;73:449-51.

58. Vuori I. Sauna bather's circulation. Ann Clin Res 1988;20:249-56.

59. Hiromitsu M, Hisashi K, Hiroyuki N, Katsunori O, Yoshihiko M, Akira M et al. Safety and efficacy of repeated sauna bathing in patients with chronic systolic heart failure: A preliminary report. J of Card Fail 2005;11:432-36.

60. Mayo Clinic Tools for Healthier Living, High Blood Pressure (Hypertension), www.mayoclinic.com/print/high-blood-pressure/DS00100/DSSECTION1.

61. Palmer R M, Ferrige A G & Moncada S (1987). Nitric oxide accounts for the biological activity of endothelium-derived relaxing factor. Nature 327, 524-526.

62. Wirth A., M. Schroeter, C. Kock-Hauser, E. Manser , J. M. Chalovich, P. de Lanerolle and G. Pfitzer. (2003)Inhibition of contraction and myosin light chain phosphorylation in guinea-pig smooth muscle by p21-activated kinase 1. J Physiol, 549.2, pp. 489-500.

63. Jugdutt B I. Nitric oxide in heart failure: friend or foe. Heart Fail Rev 2002; 7(4):385-389.

64. Tada H, Egashira K, Yamamoto M, Usui M, Arai Y, Katsuda Y et al. Role of nitric oxide in regulation of coronary blood flow in response to increased metabolic demand in dogs with pacing-induced heart failure. Jpn Circ J 2001; 65(9):827-833.

65. O'Murchu B, Miller V M, Perrella M A, Burnett J C, Jr. Increased production of nitric oxide in coronary arteries during congestive heart failure. J Clin Invest 1994; 93(1):165-171.

66. Nordhaug D, Steensrud T, Aghajani E, Korvald C, Myrmel T. Nitric oxide synthase inhibition impairs myocardial efficiency and ventriculo-arterial matching in acute ischemic heart failure. Eur J Heart Fail 2004; 6(6):705-713.

67. Datta B, Tufnell-Barrett T, Bleasdale R A, Jones CJ, Beeton I, Paul V et al. Red blood cell nitric oxide as an endocrine vasoregulator: a potential role in congestive heart failure. Circulation 2004; 109(11):1339-1342.

68. Vanderheyden M, Bartunek J, Knaapen M, Kockx M, De Bruyne B, Goethals M. Hemodynamic effects of inducible nitric oxide synthase and nitrotyrosine generation in heart failure. J Heart Lung Transplant 2004; 23(6):723-728.

69. Yoshida H, Nakamura M, Akatsu T, Arakawa N, Hiramori K. Effects of nitric oxide inhibition on basal forearm blood flow in patients with nonischemic chronic heart failure. Heart Vessels 1998; 13(3):142-146.

70. Sugahara Y, Ishii M, Muta H, Egami K, Akagi T, Matsuishi T. Efficacy and safety of thermal vasodilation therapy by sauna in infants with severe congestive heart failure secondary to ventricular septal defect. Am J Cardiol 2003; 92(1):109-113.

71. Kihara T, Biro S, Imamura M, Yoshifuku S, Takasaki K, Ikeda Y et al. Repeated sauna treatment improves vascular endothelial and cardiac function in patients with chronic heart failure. J Am Coll Cardiol 2002; 39(5):754-759.

72. Imamura M, Biro S, Kihara T, Yoshifuku S, Takasaki K, Otsuji Y et al. Repeated thermal therapy improves impaired vascular endothelial function in patients with coronary risk factors. J Am Coll Cardiol 2001; 38(4):1083-1088

73. Harris M B, Blackstone M A, Ju H, Venema V J, Venema R C. Heat-induced increases in endothelial NO synthase expression and activity and endothelial NO release. Am J Physiol Heart Circ Physiol 2003; 285(1):H333-H340

74. Ikeda Y, Biro S, Kamogawa Y, Yoshifuku S, Eto H, Orihara K et al. Repeated thermal therapy upregulates arterial endothelial nitric oxide synthase expression in Syrian golden hamsters. Jpn Circ J 2001; 65(5):434-438.

75. Ikeda Y, Biro S, Amogawa Y, Yoshifuku S, Eto H, Orihara K et al. Sauna therapy increases nitric oxide production through increasing arterial endothelial, but not inducible, nitric oxide synthase in heart failure. Journal of Cardiac Failure 10[5], S 188. 2004.

76. Gupta M, Vavasis C, Frishman W H. Heat shock proteins in cardiovascular disease a new therapeutic target. Cardiol Rev 2004; 12(1):26-30.

77. Becker J, Craig E A. Heat-shock proteins as molecular chaperones. Eur J Biochem 1994; 219(1-2):11-23.

78. Knowlton A A. The role of heat shock proteins in the heart. J Mol Cell Cardiol 1995; 27(1):121-131.

79. Ryan, Michael and Levy Mitchell M. Clinical review: Fever in intensive care unit patients. Critical Care 2003, 7:221-225.

80. Tanonaka K, Yoshida H, Toga W, Furuhama K, Takeo S. Myocardial heat shock proteins during the development of heart failure. Biochem Biophys Res Commun 2001; 283(2):520-525.

81. Tanonaka K, Toga W, Yoshida H, Takeo S. Myocardial Heat Shock Protein Changes in the Failing Heart Following Coronary Artery Ligation. Heart Lung Circ 2003; 12(1):60-65.

82. Delcayre C, Samuel J L, Marotte F, Best-Belpomme M, Mercadier J J, Rappaport L. Synthesis of stress proteins in rat cardiac myocytes 2-4 days after imposition of hemodynamic overload. J Clin Invest 1988; 82(2):460-468.

83. Tanonaka K, Toga W, Yoshida H, Furuhama K, Takeo S. Effect of long-term treatment with trandolapril on Hsp72 and Hsp73 induction of the failing heart following myocardial infarction. Br J Pharmacol 2001; 134(5):969-976.

84. Currie R W, Karmazyn M, Kloc M, Mailer K. Heat-shock response is associated with enhanced postischemic ventricular recovery. Circ Res 1988; 63(3):543-549.

85. Robinson B L, Morita T, Toft D O, Morris J J. Accelerated recovery of postischemic stunned myocardium after induced expression of myocardial heat-shock protein (HSP70). J Thorac Cardiovasc Surg 1995; 109(4):753-764.

86. Steare S E, Yellon D M. The protective effect of heat stress against reperfusion arrhythmias in the rat. J Mol Cell Cardiol 1993; 25(12):1471-1481.

87. Latchman D S. Heat shock proteins and cardiac protection. Cardiovasc Res 2001; 51(4):637-646.

88. Christians E S, Yan L J, Benjamin I J. Heat shock factor 1 and heat shock proteins: Critical partners in protection against acute cell injury. Crit Care Med 2002; 30(1 Supp):S43-S50.

89. Moskowitz R, Kukin M. Oxidative stress and congestive heart failure. Congest Heart Fail 1999; 5(4):153-163.

90. Cotran R S, Kumar V, Collins T. Robbins Pathologic Basis of Diseases. 6th ed. Philadelphia: W. B Saunders Company; 1999.

91. MC CORD, J. M. & FRIDOVICH, I. (1969). Superoxide dismutase: an enzymatic function for erythrocuprein. J. Biol. Chem., 244: 6049-6055

92. Shizukuda Y, Buttrick P M. Oxygen free radicals and heart failure: new insight into an old question. Am J Physiol Lung Cell Mol Physiol 2002; 283(2):L237-L238.

93. Dhalla A K, Singal P K. Antioxidant changes in hypertrophied and failing guinea pig hearts. Am J Physiol 1994; 266(4 Pt 2):H1280-H1285.

94. Hill M F, Singal P K. Antioxidant and oxidative stress changes during heart failure subsequent to myocardial infarction in rats. Am J Pathol 1996; 148(1):291-300.

95. McMurray J, Chopra M, Abdullah I, Smith W E, Dargie H J. Evidence of oxidative stress in chronic heart failure in humans. Eur Heart J 1993; 14(11):1493-1498.

96. Landmesser U, Spiekermann S, Dikalov S, Tatge H, Wilke R, Kohler C et al. Vascular oxidative stress and endothelial dysfunction in patients with chronic heart failure: role of xanthine-oxidase and extracellular superoxide dismutase. Circulation 2002; 106(24):3073-3078.

97. Das D K, Maulik N, Moraru I I. Gene expression in acute myocardial stress. Induction by hypoxia, ischemia, reperfusion, hyperthermia and oxidative stress. J Mol Cell Cardiol 1995; 27(1):181-193.

98. Mocanu M M, Steare S E, Evans M C, Nugent J H, Yellon D M. Heat stress attenuates free radical release in the isolated perfused rat heart. Free Radic Biol Med 1993; 15(4):459-463.

99. Steare S E, Yellon D M. The protective effect of heat stress against reperfusion arrhythmias in the rat. J Mol Cell Cardiol 1993; 25(12):1471-1481.

100. Currie R W, Karmazyn M, Kloc M, Mailer K. Heat-shock response is associated with enhanced postischemic ventricular recovery. Circ Res 1988; 63(3):543-549.

101. Karmazyn M, Mailer K, Currie R W. Acquisition and decay of heat-shock-enhanced postischemic ventricular recovery. Am J Physiol 1990; 259(2 Pt 2):H424-H431.

102. Joyeux M, Ribuot C, Bourlier V, Verdetti J, Durand A, Richard M J et al. In vitro antiarrhythmic effect of prior whole body hyperthermia: implication of catalase. J Mol Cell Cardiol 1997; 29(12):3285-3292.

103. Yellon D M, Pasini E, Cargnoni A, Marber M S, Latchman D S, Ferrari R. The protective role of heat stress in the ischaemic and reperfused rabbit myocardium. J Mol Cell Cardiol 1992; 24(8):895-907.

104. Kingma J G, Jr., Simard D, Rouleau J R, Tanguay R M, Currie R W. Effect of 3-aminotriazole on hyperthermia-mediated cardioprotection in rabbits. Am J Physiol 1996; 270(4 Pt 2):H1165-H1171.

105. Liu X, Engelman R M, Moraru I I, Rousou J A, Flack J E, III, Deaton D W et al. Heat shock.

A new approach for myocardial preservation in cardiac surgery. Circulation 1992; 86(5 Suppl):II358-II363.

106. Yamashita N, Hoshida S, Taniguchi N, Kuzuya T, Hori M. Whole-body hyperthermia provides biphasic cardioprotection against ischemia/reperfusion injury in the rat. Circulation 1998; 98(14):1414-1421.

TABLE 1
Summary of the findings of published studies on clinical impact of thermal therapy in heart failure.

| Parameters | #n of | | Thermal Therapy | | Parameter | |
|---|---|---|---|---|---|---|
| Studied | Patients | NYHA Class | Method | Parameter Change (units) | Change (%) | Ref. |
| Vascular | 20 | II-III | 2 weeks dry sauna | ↓ SVR from 2,267 ± 640 to 1,910 ± 451 dynes · s⁻¹ · m⁵ | ↓ 15.7 | (8) |
| Resistance | 32 | II-IV | Single dry sauna | ↓ SVR from 1795 ± 468 to 1205 ± 320 dynes · s⁻¹ · m⁵ during SB | ↓ 32.9 | (18) |
| | 26 | II-IV | Single warm water | ↓ SVR from 1842 ± 592 1077 ± 296 dynes · s^{-l} · m⁵ during | ↓ 41.5 | (18) |
| | | | immersion | thermal therapy | | |
| | 32 | II-IV | Single dry sauna | ↓ PVR from 238 ± 74 to 213 ± 62 | ↓ 10.5 | (18) |
| | 20 | II-III | 2 weeks dry sauna | ↑ % FMD from 4.4 ± 2.5 to 5.7 ± 2.5 | ↑ 29.5 | (8) |
| Ejection fraction | 28 | II-IV | Single dry sauna | ↑ from 24.1 ± 8.2 to 28.5 ± 8.6 | ↑ 18.3 | (18) |
| | 20 | II-IV | Single warm water | ↑ from 23.8 ± 9.5 to 29.2 ± 10.6 | ↑ 22.7 | (18) |
| | | | immersion | | | |
| | 53 | III-IV | 4 weeks warm water | ↑ from 24 ± 7% to 31 ± 9% | ↑ 29.2 | (20) |
| | | | immersion and/or | | | |
| | | | sauna bath | | | |
| Cardiac index | 32 | II-IV | Single dry sauna | ↑ from 2.7 ± 0.5 to 4.0 ± 0.7 L/min · m² | ↑ 48.1 | (18) |
| | 26 | II-IV | Single warm water | ↑ from 2.8 ± 0.5 to 4.2 ± 0.7 L/min · m² | ↑ 50 | (18) |
| | | | immersion | | | |
| Stroke index | 32 | II-IV | Single dry sauna | ↑ from 36 ± 7 to 41 ± 7 mL/min · m² | ↑ 13.9 | (18) |
| | 26 | II-IV | Single warm water | ↑ from 37 ± 7 to 43 ± 7 mL/min · m² | ↑ 16.2 | (18) |
| | | | immersion | | | |
| Diastolic Blood | 32 | II-IV | Single sauna bath | ↓ diastolic blood pressure | — | (15) |
| Pressure | 26 | II-IV | Single warm water | ↓ from 79 ± 12 to 68 ± 10 mm Hg after bath | ↓ 13.9 | (18) |
| | | | immersion | | | |
| | 32 | II-IV | Single dry sauna | ↓ from 78 ± 10 to 67 ± 11 mm Hg after bath | ↓ 14.1 | (18) |
| Systolic Blood | 20 | II-III | 2 weeks dry sauna | ↓ from 107 ± 22 to 97 ± 17 mm Hg | ↓ 9.3 | (8) |
| Pressure | 15 | II-III | 4 weeks dry sauna | ↓ from 101 ± 13 to 98 ± 14 mm Hg | ↓ 3.0 | (59) |
| Intracardiac | 34 | II-IV | Single dry sauna or | ↓ PAP, PCWP, RAP | — | (18) |
| Pressures | | | warm water immersion | | | |
| Arrhythmia | 34 | II-IV | Single dry sauna or | Noted ↓ coupled or multiform extrasystoles | — | (18) |
| | | | warm water immersion | | | |
| | 26 | II-III | 2 weeks dry sauna | ↓ PVC from 2,993 ± 905 to 1,476 ± 592/24h | ↓ 50.7 | (34) |
| | 20 | II-III | 2 weeks dry sauna | ↓ PVC from 3,161 ± ,104 to 848 ± 415/24h | ↓ 73.2 | (19) |
| | | | | ↑ HRV | | |
| Cardiac | 28 | II-IV | Single dry sauna | ↓ LV and LA dimensions | — | (18) |
| Dimensions | 20 | II-IV | Single warm water | ↓ LV and LA dimensions | — | (18) |
| | | | immersion | | | |
| | 20 | II-III | 2 weeks dry sauna | ↓ CTR from 58.2 ± 7.1 to 55.9 ± 7.9% | ↓ 4 | (8) |
| | | | | ↓ LVEDD from 59 ± 8 to 57 ± 9 mm | ↓ 3.4 | |
| | 20 | II-III | 2 weeks dry sauna | ↓ CTR from 59 ± 1 to 56 ± 2% | ↓ 5 | (19) |
| | 26 | II-III | 2 weeks dry sauna | ↓ CTR from 60 ± 2 to 57 ± 0% | ↓ 5 | (34) |
| | 56 | III-IV | 4 weeks warm water | ↓ CTR from 61 ± 5% to 55 ± 4% | ↓ 9.8 | (20) |
| | | | immersion and/or | ↓ LVEDD from 66 ± 5% to 62 ± 5 mm | ↓ 6.1 | |
| | | | sauna bath | | | |
| Mitral | 34 | II-IV | Single dry sauna or | Decreased in 20 cases out 26 with MR at baseline | — | (18) |
| Regurgitation | | | warm water immersion | | | |
| Sign and | 56 | III-IV | 4 weeks warm water | ↓ 1.2 NYHA class | — | (20) |
| Symptoms | | | immersion and/or | | | |
| | | | sauna bath | | | |
| | 15 | II-III | 6 weeks peripheral | Improved positive mood, physical capacity, relaxation, | — | (16) |
| | | | warm water baths and | socialization and depression | | |
| | | | cold applications | | | |
| | 20 | II-III | 2 weeks dry sauna | Clinical symptoms-Dyspnea, fatigue, edema, sleeplessness, | | (19) |
| | | | | appetite loss and constipation-improved in 17/20 pts. | | |
| BNP | 20 | II-III | 2 weeks dry sauna | ↓ from 425 ± 102 to 229 ± 54 pg/ml | ↓ 46.1 | (19) |
| | 26 | II-III | 2 weeks dry sauna | ↓ from 514 ± 141 to 204 ± 92 pg/ml | ↓ 60.3 | (34) |
| | 20 | II-III | 2 weeks dry sauna | ↓ from 441 ± 444 to 293 ± 302 pg/ml | ↓ 33.6 | (8) |
| NO | 12 | “infants with | 4 weeks dry sauna | ↑ from 310 to 600 μmol/l in urine | ↑ 93.5 | (22) |
| Bioavailability | | severe CHF” | | | | |

Table 1 Legend
Symbols: ↑ Increased, ↓ Decreased
Abbreviations: BNP—Brain natriuretic peptide, CHF—Congestive heart failure, CTR—Cardiothoracic ratio, DBP—Diastolic blood pressure, FMD—Flow mediated dilation, HRV—Heart rate variability, LA—Left atrium, LV—Left ventricle, LVEDD—Left ventricle end diastolic dimension, NYHA—New York Heart Association, PAP—Pulmonary artery pressure, PCWP—Pulmonary capillary wedge pressure, PVC—Premature ventricular contraction, PVR—Peripheral vascular resistance, RAP—Right atrial pressure, RV—Right ventricle, SVR—Systemic vascular resistance, VSD—Ventricular septal defect

1. A method for treating or preventing an ailment in a patient comprising:
raising a temperature of at least one portion of the patient's body by an unendangering pre-determined amount;
holding an increased temperature at the at least one portion for an unendangering pre-determined time period; and
repeating the raising and the holding until a desired result is achieved

2. The method of claim 1, wherein the temperature is raised by about 0.1° C. to about 2° C.

3. The method of claim 1, wherein the increased temperature is held for about 2 minutes to about 20 minutes.

4. The method of claim 1, wherein the raising and the holding are repeated periodically for about 1 week to about 24 weeks.

5. The method of claim 1, wherein raising the temperature is achieved by using at least one selected from the group consisting of: a heat source, a chemical and/or drug, electrical energy, electromagnetic energy, ultrasound, and radiation energy.

6. The method of claim 5, wherein the heat source is at least one selected from the group consisting of: a thermal clothing, a heat belt, a whirl pool, a sauna, a hot spring, a thermal bed, a heat pad, and ambient temperature.

7. The method of claim 1, further comprising:
administering a medication or a therapeutic device for treatment or prevention of the ailment.

8. The method of claim 7, wherein the medication or the therapeutic device is for treatment or prevention of a cardiovascular disease.

9. The method of claim 1, wherein the ailment is a cardiovascular disease.

10. The method of claim 9, wherein the cardiovascular disease is selected from the group consisting of hypertension, arrhythmia, hypertrophic disease, impaired cardiac pumping, hypertrophy of myocardium, heart failure, endothelial dysfunction, and dilation of cardiac chambers.

11. The method of claim 1, wherein the ailment is a non-cardiovascular disease.

12. The method of claim 11, wherein the non-cardiovascular disease is selected from the group consisting of: renal failure, memory loss, and obesity.

13. The method of claim 1, wherein raising the temperature comprises exposing the at least one portion to a source of increased temperature.

14. The method of claim 1, wherein the increased temperature is higher than a normal temperature for the at least one portion and lower than an endangering temperature.

15. The method of claim 1, wherein the desired result is selected from a group consisting of:
improved hemodynamics, improved ejection fractions, reduced blood pressure, increased vascular diameters, improved stroke volume, improved cardiac index, improved physical capacity, improved psychological state, and improved biochemical levels.

16. The method of claim 1, wherein the temperature is raised for the patient's entire body and the increased temperature is held for the patient's entire body.
